(19) United States
(12) Patent Application Publication
(10) Pub. No.: US 2015/0087996 A1
(43) Pub. Date: Mar. 26, 2015

(54) HOLDER AND OPTICAL BIOMETRIC APPARATUS INCLUDING THE SAME

(71) Applicant: Shimadzu Corporation, Kyoto-shi (JP)

(72) Inventors: Haruhide Udagawa, Kyoto-shi (JP); Yoshihiro Inoue, Kyoto-shi (JP); Takashi Amita, Kyoto-shi (JP); Yoshinori Masuda, Kyoto-shi (JP); Akihiro Ishikawa, Kyoto-shi (JP)

(21) Appl. No.: 14/556,223

(22) Filed: Nov. 30, 2014

Related U.S. Application Data
(63) Continuation of application No. PCT/JP2012/063869, filed on May 30, 2012.

(30) Foreign Application Priority Data
May 30, 2012 (JP) PCT/JP2012/063869

(51) Int. Cl. A61B 5/00

(52) U.S. Cl. CPC A61B 5/0075; A61B 5/0082; A61B 5/4064
USPC 600/473

(57) ABSTRACT

A holder configured to allow light transmission probes for emitting light and light reception probes for receiving light to be arranged alternately and be spaced apart from each other by a second set distance r₂. The holder has a plurality of first through holes each provided at a position spaced apart by a first set distance r₁ shorter than the second set distance r₂ from a position of the retained light transmission probe or a position of the retained light reception probe. Each of the through holes of the plurality of first through holes allow reference probes for emitting or receiving light to be inserted thereto. One of the first through holes provided therein with none of the reference probes has a non-light transmissive attachment member in a detachable manner.

### CROSS-REFERENCE TO RELATED APPLICATION

This application claims priority to and is a continuation of PCT Application No. PCT/JP2012/063869, filed May 30, 2012, the entire contents of which are incorporated herein by reference.

### TECHNICAL FIELD

The present disclosure relates to a holder and an optical biometric apparatus including the same.

### BACKGROUND

Recently, an optical brain function imaging apparatus (optical biometric apparatus) has been developed that easily measures using light in a non-invasive manner for observation of brain activity conditions. Such an optical brain function imaging apparatus includes a light transmission probe, which is disposed on a scalp surface of an examinee and irradiates a brain with near infrared light rays having three different wavelengths λ₁, λ₂, and λ₃ (e.g. 780 nm, 805 nm, and 830 nm), and a light reception probe, which is disposed on the scalp surface and detects intensity changes (received light amount information pieces) ΔA(λ₁), ΔA(λ₂), and ΔA(λ₃) of the near infrared light rays having the wavelengths λ₁, λ₂, and λ₃ radiated from the brain.

In order to find a product of a concentration change and an optical path length of oxyhemoglobin [oxyHb] and a product of a concentration change and an optical path length of deoxyhemoglobin [deoxyHb] in a cerebral blood flow from the received light amount information pieces ΔA(λ₁), ΔA(λ₂), and ΔA(λ₃) thus obtained, the apparatus creates simultaneous equations including relational equations (1), (2), and (3) according to the Modified Beer Lambert law or the like, and solves these simultaneous equations. The apparatus further calculates a product of a concentration change and an optical path length of total hemoglobin ([oxyHb]+[deoxyHb]) from the product of the concentration change and the optical path length of oxyhemoglobin [oxyHb] and the product of the concentration change and the optical path length of deoxyhemoglobin [deoxyHb].

ΔA(λ₁)=E₀(λ₁)×[oxyHb]+E_{d}(λ₁)×[deoxyHb] (1)

ΔA(λ₂)=E₀(λ₂)×[oxyHb]+E_{d}(λ₂)×[deoxyHb] (2)

ΔA(λ₃)=E₀(λ₃)×[oxyHb]+E_{d}(λ₃)×[deoxyHb] (3)

In these relational equations, E₀(λ m) denotes an absorbance coefficient of oxyhemoglobin for light of a wavelength λ m and E_{d}(λ m) denotes an absorbance coefficient of deoxyhemoglobin for the light of the wavelength λ m.

Described below is the relation of a distance (channel) between a light transmission probe and a light reception probe, to a measured site. FIGS. 6(a) and 6(b) are views showing the relation of a light transmission probe and a light reception probe in a pair, to a measured site. A light transmission probe 12 is pressed to a light transmission point T on a scalp surface of an examinee whereas a light reception probe 13 is pressed to a light reception point R on the scalp surface of the examinee. Light is emitted from the light transmission probe 12 whereas light radiated from the scalp surface is made incident on the light reception probe 13. At this time, light passed through a banana shape (measured region) out of the light emitted from the light transmission point T on the scalp surface reaches the light reception point R on the scalp surface. For example, the light will pass through a blood vessel in a skin around the light transmission point T, a blood vessel in the brain, and a blood vessel in a skin around the light reception point R.

For obtaining received light amount information ΔA only on the blood vessel in the brain, an apparatus that includes a light transmission probe 12 and a light reception probe 13 having a short distance r₁ as the distance (channel) therebetween as well as a light transmission probe 12 and a light reception probe 13 having a long distance r₂ as the distance (channel) therebetween may be used (see Patent Document 1, Non-Patent Document 1, and the like). FIG. 7 is a sectional view showing the relation of a reference probe 14 spaced apart by the short distance r₁ from the light transmission probe 12 and the light reception probe 13 spaced apart by the long distance r₂ from the light transmission probe 12, to measured sites. Second received light amount information ΔA2 on the blood vessel in the skin around the light transmission point T, the blood vessel in the brain, and a blood vessel in a skin around a light reception point R2 is obtained in the channel of the long distance r₂ whereas first received light amount information ΔA1 only on the blood vessel in the skin around the light transmission point T (the blood vessel in the skin around a light reception point R1) is obtained in the channel of the short distance r₁.

The received light amount information ΔA only on the blood vessel in the brain is found from the received light amount information pieces ΔA1 and ΔA2 thus obtained in accordance with an equation (4).

ΔA=ΔA2−KΔA1 (4)

In these systems, it is generally necessary to determine a coefficient K for finding the received light amount information ΔA in accordance with the equation (4). A method of calculating this coefficient K is disclosed (see Non-Patent Document 2, for example). The coefficient K is calculated with reference to a minimum square error in this calculation method.

The optical brain function imaging apparatus further includes a near-infrared spectral analyzer or the like for measuring a product of a concentration change and an optical path length of oxyhemoglobin [oxyHb], a product of a concentration change and an optical path length of deoxyhemoglobin [deoxyHb], and a product of a concentration change and an optical path length of total hemoglobin ([oxyHb]+[deoxyHb]) at each of a plurality of measured sites in a brain (see Patent Document 2, for example).

Such a near-infrared spectral analyzer includes a holder 130 for causing the light transmission probes 12, the light reception probes 13, and the reference probes 14 to be in contact with a scalp surface of an examinee in a predetermined arrangement. FIG. 8 is a plan view exemplifying the holder 130 that allows eight light transmission probes 12, eight light reception probes 13, and twelve reference probes 14 to be inserted thereto.

The holder 130 has second through holes T1 to T8 and R1 to R8 that allow eight light transmission probes 12_{T1} to 12_{T8} and eight light reception probes 13_{R1} to 13_{R8} to be inserted thereto, and first through holes B1 to B12 that allow twelve reference probes 14_{B1} to 14_{B12} to be inserted thereto.

The second through holes T1 to T8 allowing the light transmission probes 12_{T1} to 12_{T8} to be inserted thereto and the second through holes R1 to R8 allowing the light reception probes 13_{R1} to 13_{R8} to be inserted thereto are formed to be arranged alternately in a square lattice shape of four probes in the longitudinal direction and four probes in the transverse direction. In this case, each of the second through holes T1 to T8 allowing the light transmission probes 12_{T1} to 12_{T8} to be inserted thereto and an adjacent one of the second through holes R1 to R8 allowing the light reception probes 13_{R1} to 13_{R8} to be inserted thereto have a second set distance r₂, as a gap (channel) therebetween, of 30 mm. The apparatus can thus collect second received light amount information pieces ΔA2ₙ(λ₁), ΔA2ₙ(2₂), and ΔA2ₙ(λ₃) (n=1, 2, . . . , 24) on 24 measured positions.

The first through hole B1 allowing the reference probe 14_{B1} to be inserted thereto is formed between the second through hole T1 allowing the light transmission probe 12_{T1} to be inserted thereto and the second through hole R3 allowing the light reception probe 13_{R3} to be inserted thereto, at a position distant by a first set distance r₁ from the second through hole T1 allowing the light transmission probe 12_{T1} to be inserted thereto. The first set distance r₁, as a gap between the second through hole T1 allowing the light transmission probe 12_{T1} to be inserted thereto and the first through hole B1 allowing the reference probe 14_{B1} to be inserted thereto, is 15 mm. The first through hole B2 allowing the reference probe 14_{B2} to be inserted thereto is formed at a position distant by the first set distance r₁ from the first through hole T3 allowing the light transmission probe 12_{T3} to be inserted thereto, and the first through hole B3 allowing the reference probe 14_{B3} to be inserted thereto is formed at a position distant by the first set distance r₁ from the second through hole T2 allowing the light transmission probe 12_{T2} to be inserted thereto. In these manners, the first through holes allowing the reference probes 14 to be inserted thereto are each formed at a position distant by the first set distance r₁ from the corresponding one of the second through holes allowing the light transmission probes 12 to be inserted thereto. The apparatus can thus collect first received light amount information pieces ΔA1ₘ(λ₁), ΔA1ₘ(λ₂), and ΔA1ₘ(λ₃) (m=1, 2, . . . , 12) on twelve measured positions.

### PRIOR ART DOCUMENTS

The following documents are provided for reference, and are incorporated herein by reference in their entirety.

### Patent Documents

Patent Document 1: JP 2009-136434 A

Patent Document 2: JP 2001-337033 A

### Non-Patent Documents

Non-Patent Document 1: Rolf B. Saager, and Andrew J. Berger “Direct characterization and removal of interfering absorption trends in two-layer turbid media” J. Opt. Soc. Am. A/Vol. 22, No. 9/September 2005.

Non-Patent Document 2: Francesco Fabbri, Angelo Sassaroli, Michael e Henry, and Sergio Fantini “Optical measurements of absorption changes in two-layered diffusive media” Phys. Med. Biol. 49(2004) 1183-1201.

### SUMMARY

According to certain embodiments, a holder is configured to allow light transmission probes for emitting light and light reception probes for receiving light to be arranged alternately and be spaced apart from each other by a second set distance r₂. The holder has a plurality of first through holes each provided at a position spaced apart by a first set distance r₁ shorter than the second set distance r₂ from a position of the retained light transmission probe or a position of the retained light reception probe. Each of the through holes of the plurality of first through holes allow reference probes for emitting or receiving light to be inserted thereto. One of the first through holes provided therein with none of the reference probes has a non-light transmissive attachment member in a detachable manner.

### BRIEF DESCRIPTION OF THE DRAWINGS

FIG. 1 is a block diagram showing the schematic configuration of an optical biometric apparatus according an embodiment of the present invention.

FIG. 2 is a plan view exemplifying a holder, which allows eight light transmission probes, eight light reception probes, and twelve reference probes to be inserted thereto, having twelve attachment members being inserted thereto, according to one exemplary embodiment.

FIGS. 3(a) and 3(b) are perspective views each exemplifying the attachment member, according to example embodiments.

FIG. 4 is a plan view exemplifying the holder that has eight light transmission probes, eight light reception probes, and four attachment members 40 being inserted thereto, according to one exemplary embodiment.

FIG. 5 is a flowchart illustrating an exemplary method of using the holder.

FIGS. 6(a) and 6(b) are views showing the relation of a light transmission probe and a light reception probe in a pair, to a measured site.

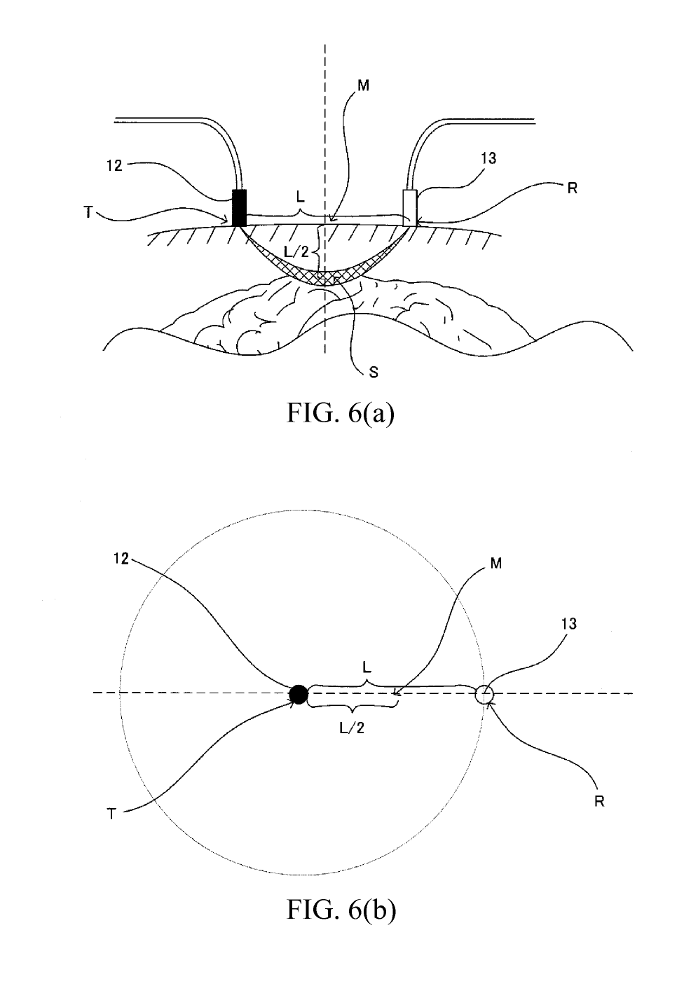

FIG. 7 is a sectional view showing the relation of a reference probe distant by a short distance r₁ from the light transmission probe and the light reception probe distant by a long distance r₂ from the light transmission probe, to measured sites.

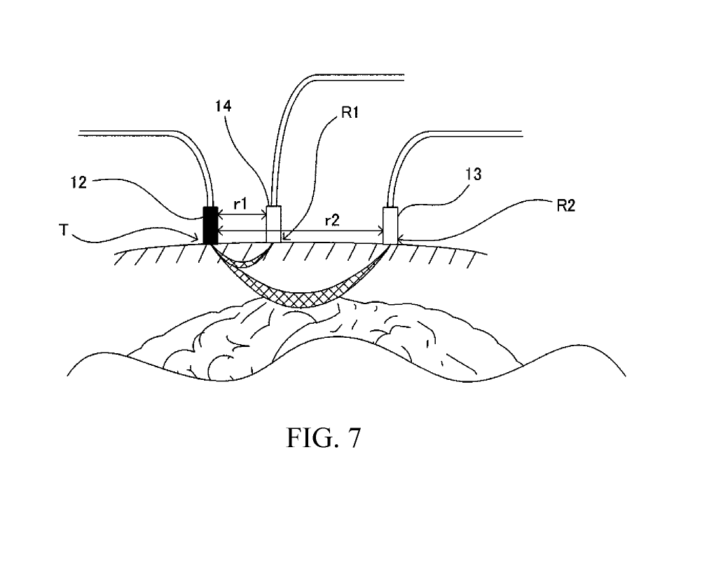

FIG. 8 is a plan view exemplifying the holder that allows eight light transmission probes, eight light reception probes, and twelve reference probes to be inserted thereto.

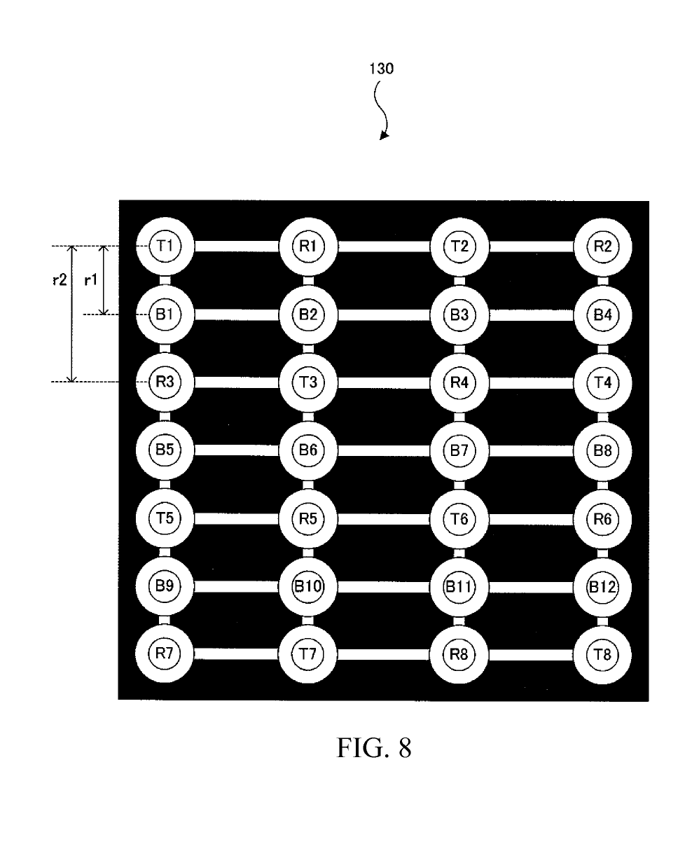

### DETAILED DESCRIPTION

The present disclosure now will be described more fully hereinafter with reference to the accompanying drawings, in which various embodiments are shown. The invention may, however, be embodied in many different forms and should not be construed as limited to the example embodiments set forth herein. It should also be emphasized that the disclosure provides details of alternative examples, but such listing of alternatives is not exhaustive. Furthermore, any consistency of detail between various examples should not be interpreted as requiring such detail—it is impracticable to list every possible variation for every feature described herein. The language of the claims should be referenced in determining the requirements of the invention.

In the drawings, the size and relative sizes of components and regions may be exaggerated for clarity. Like numbers refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. As used herein, the term “and/or” includes any and all combinations of one or more of the associated listed items and may be abbreviated as “/”.

It will be understood that, although the terms first, second, etc. may be used herein to describe various elements, these elements should not be limited by these terms. Unless indicated otherwise, these terms are only used to distinguish one element from another, for example as a naming convention. For example, a first device could be termed a second device, and, similarly, a second device could be termed a device without departing from the teachings of the disclosure.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that when an element is referred to as being “connected” or “coupled” to or “on” another element, it can be directly connected or coupled to or on the other element or intervening elements may be present. In contrast, when an element is referred to as being “directly connected” or “directly coupled” to another element, or as “contacting” another element, there are no intervening elements present. Other words used to describe the relationship between elements should be interpreted in a like fashion (e.g., “between” versus “directly between,” “adjacent” versus “directly adjacent,” etc.).

Embodiments described herein will be described referring to plan views and/or cross-sectional views by way of ideal schematic views. Accordingly, the exemplary views may be modified depending on manufacturing technologies and/or tolerances. Therefore, the disclosed embodiments are not limited to those shown in the views, but include modifications in configuration formed on the basis of manufacturing processes. Therefore, regions exemplified in figures may have schematic properties, and shapes of regions shown in figures may exemplify specific shapes of regions of elements to which aspects of the invention are not limited.

Spatially relative terms, such as “beneath,” “below,” “lower,” “above,” “upper” and the like, may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly.

Terms such as “same,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to reflect this meaning.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and/or the present application, and will not be interpreted in an idealized or overly formal sense unless expressly so defined herein.

The above holder 130 having the twelve first through holes B1 to B12 is used with the reference probes 14_{B1} to 14_{B12} being inserted to all of the twelve first through holes B1 to B12, or is used with the reference probes 14 being inserted to only four of the first through holes B, for example. In the latter case, ambient light is incident through any one of the first through holes B with no reference probe 14 being inserted thereto, and the light reception probes 13_{R1} to 13_{R8} may thus problematically detect the ambient light.

The above holder 130 is to be used with the eight light transmission probes 12, the eight light reception probes 13, and the four reference probes 14 being inserted thereto, while the holder 130 has these many through holes T1 to T8, R1 to R8, and B1 to B12. It is accordingly difficult to find which probe is to be inserted to which through hole, and insertion may take time or may not be performed correctly.

In view of the above, an object of the present embodiments is to prevent incidence of ambient light through any one of the first through holes as well as provide a holder that allows a probe to be inserted easily and accurately to a through hole and an optical biometric apparatus including the holder.

According to certain embodiments, a holder is configured to allow light transmission probes for emitting light and light reception probes for receiving light to be arranged alternately and be distant from each other by a second set distance r₂, wherein the holder has a plurality of first through holes each provided at a position distant by a first set distance r₁ shorter than the second set distance r₂ from a position of the retained light transmission probe or a position of the retained light reception probe, the first through holes allow reference probes for emitting or receiving light to be inserted thereto, the first through hole provided therein with none of the reference probes allows a non-light transmissive attachment member to be detachable therefrom, and the holder includes the attachment member.

The “second set distance r₂” is provided for obtaining second received light amount information on a blood vessel in a skin around a light transmission point T, a blood vessel in a brain, and a blood vessel in a skin around a light reception point R. The “first set distance r₁” is provided for obtaining first received light amount information on a blood vessel in the skin around the light transmission point T or the light reception point R.

As described above, in the holder according to the present embodiments, the first through hole with no reference probe being inserted thereto has the non-light transmissive attachment member attached thereto. The holder can thus prevent incidence of ambient light through the first through hole.

Further, when the light transmission probes and the light reception probes are attached to the holder, none of the light transmission probes and the light reception probes are erroneously inserted to the first through hole with the attachment member being attached thereto. In certain embodiments, the light transmission probes and the light reception probes have only to be inserted alternately to the through holes having no attachment members. The light transmission probes and the light reception probes can be thus inserted easily and accurately. When the reference probe is attached to the holder, the reference probe can be also inserted easily and accurately by detaching the attachment member from a desired first through hole.

In the holder according to certain embodiments, the holder has a plurality of second through holes allowing the light transmission probes or the light reception probes to be inserted thereto, and the second through hole provided therein with neither the light transmission probe nor the light reception probe allows the attachment member to be detachable therefrom.

In the holder according to certain embodiments, the first through holes can be each formed at a median of a line connecting a position of the retained light transmission probe and a position of the retained light reception probe.

The holder according to certain embodiments would allow incidence of ambient light on a measured position of a brain without any attachment member, but can prevent incidence of ambient light because the attachment member is provided.

In the holder according to certain embodiments, the second set distance r₂ can be 30 mm. However, other distances may be used.

An optical biometric apparatus according to the certain embodiments can include the holder as described above, a light transmission probe for emitting light, a light reception probe for receiving light, a reference probe for emitting or receiving light, and a controller for controlling light transmission or light reception of the light transmission probe, the light reception probe, or the reference probe.

An exemplary embodiment will now be described below with reference to the drawings. The present invention is not limited to the following embodiment, but includes various aspects within the range consistent with the spirit and scope of the present disclosure.

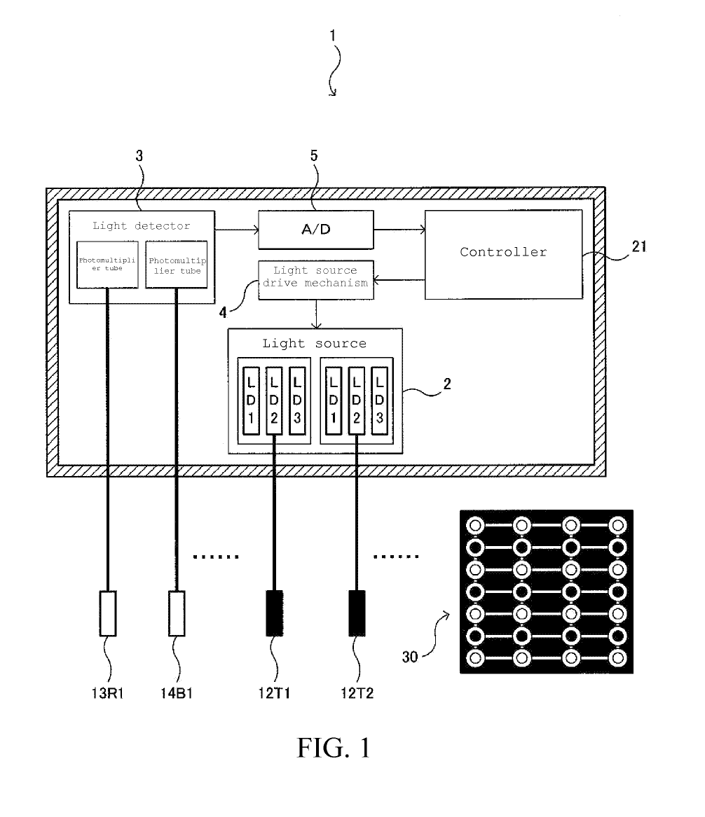

FIG. 1 is a block diagram showing the schematic configuration of an optical biometric apparatus according an embodiment of the present invention. An optical biometric apparatus 1 includes a light source 2 for emitting light, a light source drive mechanism 4 for driving the light source 2, a light detector 3 for detecting light, an A/D converter (A/D) 5, a controller 21, as well as eight light transmission probes 12, eight light reception probes 13, four reference probes 14, and a holder 30.

The light source drive mechanism 4 transmits light to one of the light transmission probes 12 selected from the eight light transmission probes 12_{T1} to 12_{T8} in accordance with a drive signal received from the controller 21. The light is exemplified by near infrared light (e.g. light rays having three wavelengths of 780 nm, 805 nm, and 830 nm).

The light detector 3 individually detects near infrared light rays (e.g. light rays having three wavelengths of 780 nm, 805 nm, and 830 nm) received by the eight light reception probes 13_{R1} to 13_{R8} to transmit eight pieces of second received light amount information ΔA2(λ₁), ΔA2(λ₂), and ΔA2(λ₃) to the controller 21. The light detector 3 also individually detects near infrared light rays (e.g. light rays having three wavelengths of 780 nm, 805 nm, and 830 nm) received by the four reference probes 14 to transmit four pieces of first received light amount information ΔA1(λ₁), ΔA1(λ₂), and ΔA1(λ₃) to the controller 21.

The light transmission probes 12 each have a columnar shape so as to be inserted to a second through hole T. The light transmission probes 12 each have an upper end connected to the light source 2 by way of a light guiding path such as an optical fiber, as well as a lower end for emitting light.

The light reception probes 13 each have a columnar shape similar to that of the light transmission probe 12. The light reception probes 13 each have an upper end connected to the light detector 3 by way of a light guiding path such as an optical fiber, and a lower end for receiving light.

The reference probes 14 each have a columnar shape similar to that of the light transmission probe 12. The light reception probes 13 each have an upper end connected to the light detector 3 by way of a light guiding path such as an optical fiber, and a lower end for receiving light.

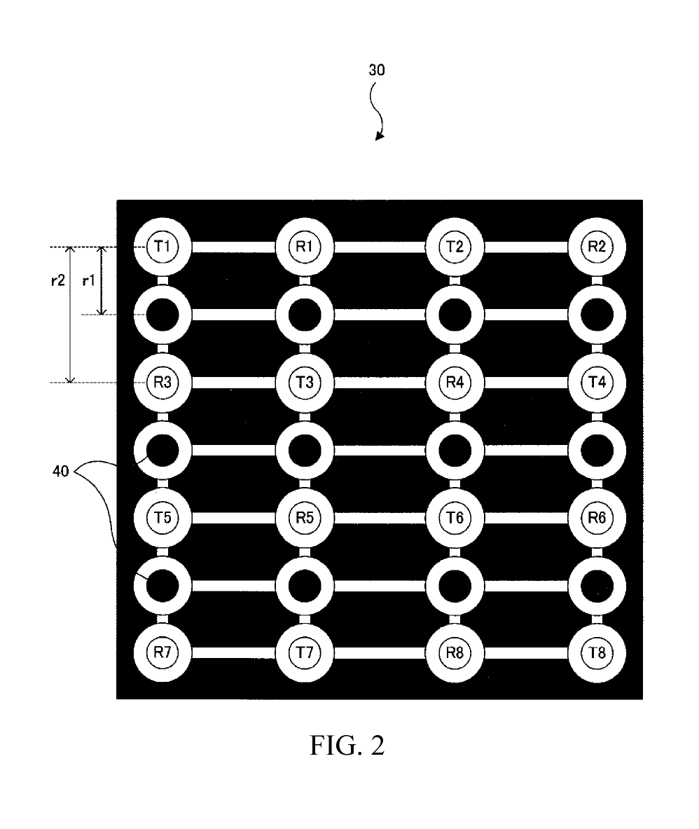

FIG. 2 is a plan view exemplifying the holder 30, which allows the eight light transmission probes 12, the eight light reception probes 13, and the twelve reference probes 14 to be inserted thereto, having twelve attachment members 40 being inserted thereto. The components configured similarly to those of the holder 130 are denoted by the same reference signs.

The holder 30 has second through holes T1 to T8 and R1 to R8 that allow eight light transmission probes 12_{T1} to 12_{T8} and eight light reception probes 13_{R1} to 13_{R8} to be inserted thereto, and first through holes B1 to B12 that allow twelve reference probes 14_{B1} to 14_{B12} to be inserted thereto.

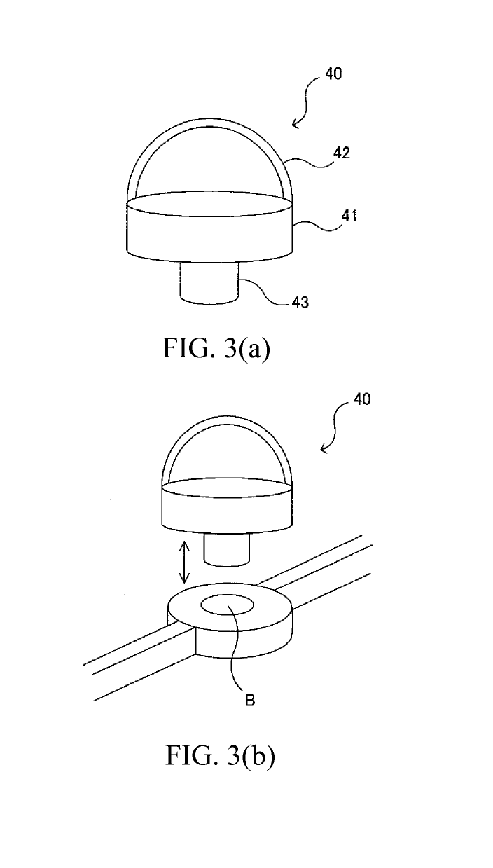

The holder 30 according to certain embodiments includes the twelve attachment members 40. However, other numbers of attachment members may be used. FIG. 3(a) is a perspective view exemplifying one of the attachment members 40. The attachment member 40 has a columnar main body 41, a grip portion 42 formed at the upper surface of the main body 41, and a columnar insert portion 43 formed at the lower surface of the main body 41.

The insert portion 43 can be inserted to the first through hole B and be extracted from the first through hole B having the insert portion 43 therein, in other words, is detachable (see FIG. 3(b)). Specifically, the insert portion 43 may have the shape same as those of the first through holes B1 to B12 or slightly larger. In a case where the first through holes B1 to B12 each have a columnar shape of 5 mm in diameter and 1 cm in depth, the insert portion 43 has a columnar shape of 5 mm in diameter and 1 cm in depth, for example. The depths may not be equal to each other.

The main body 41 preferably has the columnar shape with a diameter equal to that of a ring portion at the peripheral edge of each of the first through holes B1 to B12.

The grip portion 42 is gripped by a physician, a laboratory technician, or the like, and is used for binding the light guiding paths such as optical fibers connected to the light transmission probes 12 and the like.

The main body and the grip portion are not particularly limited in terms of their materials, and can be made of, for example, polypropylene, polyvinyl chloride, polyacetal, or the like. The insert portion is not particularly limited in terms of its material, and can be made of, for example, rubber or the like.

In one embodiment, at least one of the main body and the insert portion is be made of a non-light transmissive material. In one embodiment, both the main body and the insert portion are made of a non-light transmissive material.

The attachment member 40 thus configured can be pressed into the first through hole B from above so as to be attached to the first through hole B, and can be extracted upward from the first through hole B so as to be detached from the first through hole B.

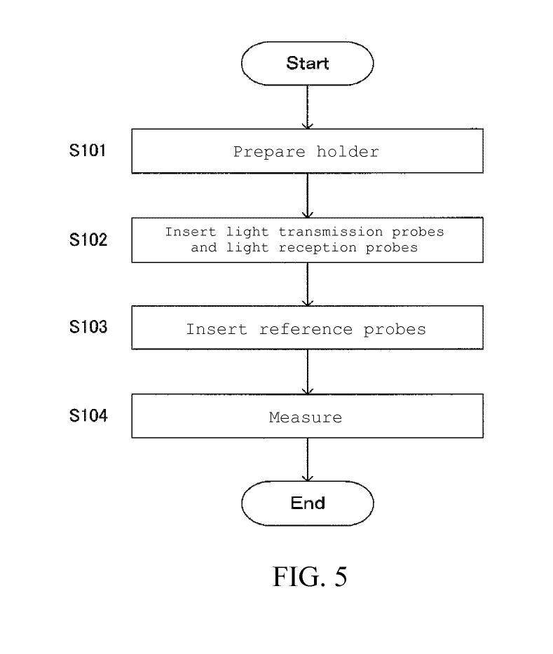

An exemplary method of using the holder 30 according to the disclosed embodiments is described next. FIG. 5 is a flowchart illustrating an exemplary method of using the holder 30.

Initially in the process in step S101, a physician, a laboratory technician, or the like, prepares the holder 30 shown in FIG. 2. At this stage, the attachment members are attached to the twelve first through holes B1 to B12, respectively.

Subsequently in the process in step S102, the physician, the laboratory technician, or the like inserts the eight light transmission probes 12_{T1} to 12_{T8} to the second through holes T1 to T8 and inserts the eight light reception probes 13_{R1} to 13_{R8} to the second through holes R1 to R8. In this state, the twelve first through holes B1 to B12 are provided with the attachment members 40, respectively. Neither the light transmission probes 12 nor the light reception probes 13 are thus erroneously inserted to the first through holes B1 to B12. Further, the light transmission probes 12 and the light reception probes 13 have only to be inserted alternately. The light transmission probes 12 and the light reception probes 13 can be thus inserted easily and accurately.

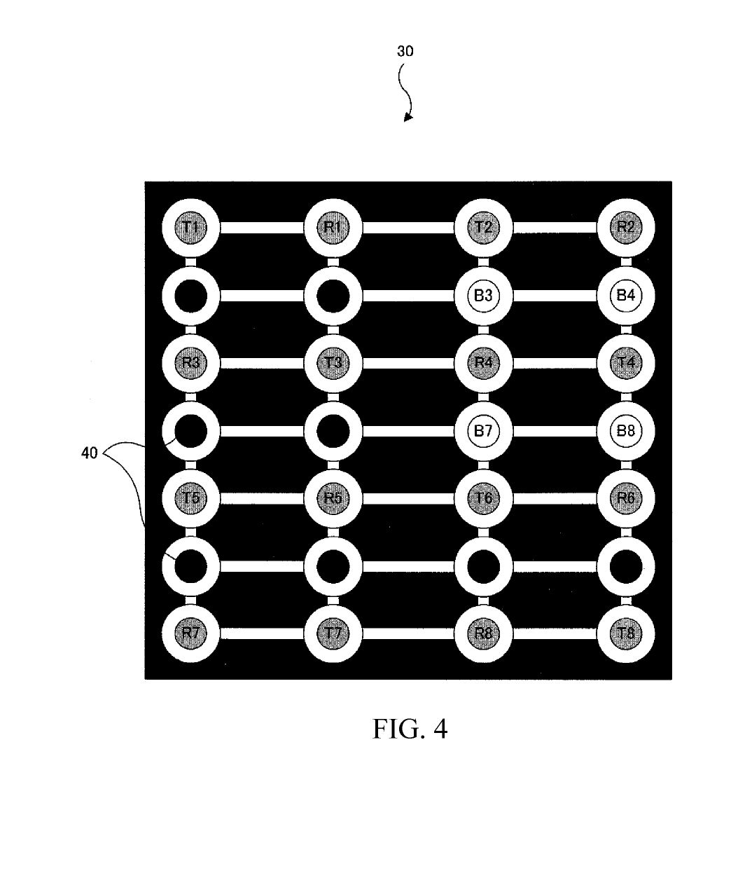

Subsequently in the process in step S103, the physician, the laboratory technician, or the like detaches the attachment members 40 from the desired four first through holes B3, B4, B7, and B8, and inserts the four reference probes 14 to the desired four first through holes B3, B4, B7, and B8. FIG. 4 is a plan view exemplifying the holder 30 that has the eight light transmission probes 12, the eight light reception probes 13, and the four attachment members 40 being inserted thereto. Note that the light guiding paths such as optical fibers connected to the light transmission probes 12 and the like are not illustrated in the figure.

Subsequently in the process in step S104, the physician, the laboratory technician, or the like, starts measurement. The apparatus thus collects second received light amount information pieces ΔA2ₙ(λ₁), ΔA2ₙ(λ₂), and ΔA2ₙ(λ₃) (n=1, 2, . . . , 24) on 24 measured positions, as well as collects first received light amount information pieces ΔA1ₘ(λ₁), ΔA1ₘ(λ₂), and ΔA1ₘ(λ₃) (m=1, 2, . . . , 4) on four measured positions.

At this stage, the first through holes B1, B2, B5, B6, and B9 to B12, to which no reference probe 14 is inserted, are provided with the attachment members 40, respectively. Ambient light can be thus prevented from being incident through the first through holes B1, B2, B5, B6, and B9 to B12.

Then, the flow in this chart is ended upon completion of the process in step S104.

### OTHER EMBODIMENTS

Although various amounts of components and elements are described above, these are given only as examples. Other amounts of through holes, attachment members, and measured positions, for example, may be used to implement the various methods described herein.

In addition, in the optical biometric apparatus 1 described above, the twelve attachment members 40 are configured identically. Alternatively, the attachment members can be each provided with labels having probe numbers or the like so as to be identified separately from one another.

In the optical biometric apparatus 1 described above, the attachment members 40 are inserted to the first through holes B1 to B12. Alternatively, the attachment member can be inserted to any one of the second through holes T1 to T8 and R1 to R8.

In the optical biometric apparatus 1 described above, the attachment members 40 are inserted to the first through holes B so as to be detachable in a pressed manner. Alternatively, the attachment members can be each provided at the outer peripheral surface of the insert portion with a thread and the first through holes B can be each provided at the inner peripheral surface with a thread so that the attachment members are detachable in a screwed manner.

In the optical biometric apparatus 1 described above, the attachment members 40 are inserted to the first through holes B so as to be detachable in a pressed manner. Alternatively, the disc-shaped lower surface of each of the attachment members can be affixed, with an adhesive, to the upper surface of the ring portion at the peripheral edge of the first through hole B so that the attachment members are detachable in an affixed manner.

In the optical biometric apparatus 1 described above, the attachment members 40 each have the columnar main body 41, the grip portion 42, and the columnar insert portion 43. The attachment members have only to be configured so as not to allow incidence of light through the first through holes B. For example, the attachment members 40 can each have a shape without the grip portion 42, a shape without the grip portion 42 and the main body 41, or a cap shape so as to surround the ring portion at the peripheral edge of the first through hole B, or have a cotton-like body to be inserted to the first through hole B.

In the optical biometric apparatus 1 described above, the single attachment member 40 is attached to one of the first through holes B. Alternatively, the single attachment member can be attached to a plurality, e.g. two, of the first through holes B.

Other variations may be implemented as well.

As described above, the disclosed embodiments can be utilized in an medical apparatus such as an optical biometric apparatus or the like for measuring brain activities in a non-invasive manner. Though not described in detail above, the apparatus may also be used for other medical purposes.

The controller and other components of the apparatus described herein may include circuitry, hardware, software, firmware, or combinations thereof. Furthermore the various actions described herein may be implemented using this circuitry, hardware, software, firmware, etc.

While the disclosure has been described with reference to example embodiments, it will be apparent to those skilled in the art that various changes and modifications may be made without departing from the spirit and scope of the disclosed embodiments. Therefore, it should be understood that the above embodiments are not limiting, but illustrative, and the scope of the invention is to be determined by the broadest permissible interpretation of the following claims and their equivalents, and shall not be restricted or limited by the foregoing description.

We claim:

1. A holder configured to allow light transmission probes for emitting light and light reception probes for receiving light to be arranged alternately and be spaced apart from each other by a second set distance r₂, wherein
the holder has a plurality of first through holes each provided at a position spaced apart by a first set distance r₁ shorter than the second set distance r₂ from a position of a retained light transmission probe or a position of a retained light reception probe,
each of the through holes of the plurality of first through holes allow reference probes for emitting or receiving light to be inserted thereto,
one of the first through holes provided with none of the reference probes allows a non-light transmissive attachment member to be detachable therefrom, and
the holder includes the attachment member.

2. The holder according to claim 1, wherein the first through holes are each formed at a median of a line connecting a position of the retained light transmission probe and a position of the retained light reception probe.

3. The holder according to claim 1, wherein the second set distance r₂ is 30 mm.

4. An optical biometric apparatus comprising:
the holder according to claim 1;
a light transmission probe for emitting light;
a light reception probe for receiving light;
a reference probe for emitting or receiving light; and
a controller for controlling light transmission or light reception of the light transmission probe, the light reception probe, or the reference probe.

5. The holder according to claim 1, wherein
the holder has a plurality of second through holes allowing the light transmission probes or the light reception probes to be inserted thereto, and
one of the second through holes provided with neither the light transmission probe nor the light reception probe allows the attachment member to be detachable therefrom.

6. The holder according to claim 5, wherein the first through holes are each formed at a median of a line connecting a position of the retained light transmission probe and a position of the retained light reception probe.

7. The holder according claim 5, wherein the second set distance r₂ is 30 mm.

8. An optical biometric apparatus comprising:
the holder according to claim 5;
a light transmission probe for emitting light;
a light reception probe for receiving light;
a reference probe for emitting or receiving light; and
a controller for controlling light transmission or light reception of the light transmission probe, the light reception probe, or the reference probe.

9. The holder according to claim 1, wherein the first through holes are each formed at a median of a line connecting a position of the retained light transmission probe and a position of the retained light reception probe, wherein the second set distance r₂ is 30 mm.

11. An optical biometric apparatus comprising:
the holder according to claim 1, wherein the first through holes are each formed at a median of a line connecting a position of the retained light transmission probe and a position of the retained light reception probe;
a light transmission probe for emitting light;
a light reception probe for receiving light;
a reference probe for emitting or receiving light; and
a controller for controlling light transmission or light reception of the light transmission probe, the light reception probe, or the reference probe.

12. An optical biometric apparatus comprising:
the holder according to claim 1, wherein the second set distance r₂ is 30 mm;
a light transmission probe for emitting light;
a light reception probe for receiving light;
a reference probe for emitting or receiving light; and
a controller for controlling light transmission or light reception of the light transmission probe, the light reception probe, or the reference probe.
